(19) United States
(12) Patent Application Publication
(10) Pub. No.: US 2026/0013767 A1
(43) Pub. Date: Jan. 15, 2026

(54) ADHESIVE ELECTRODE FOR BIOSIGNAL ACQUISITION AND BIOSENSOR

(71) Applicant: NITTO DENKO CORPORATION, Ibaraki-shi, Osaka (JP)

(72) Inventors: Keisuke HIRANO, Ibaraki-shi, Osaka (JP); Keine NISHIYAMA, Ibaraki-shi, Osaka (JP)

(21) Appl. No.: 19/114,572

(22) PCT Filed: Sep. 13, 2023
(86) PCT No.: PCT/JP2023/033311

§ 371 Date: Mar. 24, 2025

(30) Foreign Application Priority Data
Sep. 27, 2022 (JP) 2022-153422

(51) Int. Cl. A61B 5/257; C09J 139/06

(52) U.S. Cl. CPC A61B 5/257; C09J 139/06; C09J 2203/326

(57) ABSTRACT

An adhesive electrode for biosignal acquisition according to the present invention includes a conductive polymer, an aqueous emulsion adhesive, and a nitrogen-containing additive. An amount of the nitrogen-containing additive is from 0.01 wt % to 3.0 wt % relative to a total amount of the adhesive electrode for biosignal acquisition.

### TECHNICAL FIELD

The present invention relates to adhesive electrodes for biosignal acquisition and biosensors.

### BACKGROUND ART

Wearable biosensors, which acquire biological information, such as electrocardiogram waveforms, pulse waves, electroencephalograms, electromyograms, and the like are used in medical institutes, such as hospitals, clinics, or the like, nursing care facilities, homes, and the like. A biosensor includes an adhesive electrode for biosignal acquisition (bioelectrode) that is brought into contact with a body of a biological organism to acquire biological information of a subject. At the time when the biological information is measured, the biosensor is adhered to the skin of the subject and acquires electric signals associated with the biological information, thereby measuring the biological information.

As such a bioelectrode for a biosensor, for example, there is disclosed an adhesive sheet that includes an adhesive layer including a conductive organic polymer compound and an adhesive material. As the conductive organic polymer compound, PEDOT:PSS is used. As the adhesive material, an aqueous emulsion is used. The adhesive sheet is adhered to a surface of a wiring board, which is arranged to face the skin of a subject (see, for example, Patent Document 1).

### CITATION LIST

### Patent Document

Patent Document 1: Japanese Unexamined Patent Application Publication No. 2020-147659

### SUMMARY OF THE INVENTION

### Technical Problem

However, an aqueous emulsion used as an adhesive material has characteristics such that the aqueous emulsion readily absorbs moisture. As the adhesive sheet of Document 1 absorbs the sweat or moisture present in the surroundings when the adhesive sheet is adhered onto the skin, the adhesive strength is reduced so that the adhesive sheet is likely to be peeled off from the skin, and biosignals cannot be stably measured with high sensitivity.

One aspect of the present invention has an object to provide an adhesive electrode for biosignal acquisition that can perform stable measurement with high sensitivity even when external moisture is absorbed during use.

### Solution to the Problem

One aspect of the adhesive electrode for biosignal acquisition according to the present invention includes a conductive polymer, an aqueous emulsion adhesive, and a nitrogen-containing additive. An amount of the nitrogen-containing additive is from 0.01 wt % to 3.0 wt % relative to a total amount of the adhesive electrode for biosignal acquisition.

### Effects of the Invention

One aspect of the adhesive electrode for biosignal acquisition according to the present invention can perform stable measurement with high sensitivity even when external moisture is absorbed during use.

### BRIEF DESCRIPTION OF THE DRAWINGS

FIG. 1 is a graph depicting a relationship between a water absorption rate and contact impedance of the electrode sheet of each of Examples and Comparative Examples.

FIG. 2 is a graph depicting a relationship between a water absorption rate and an adhesive strength of the electrode sheet of each of Examples and Comparative Examples.

### DETAILED DESCRIPTION OF THE EMBODIMENTS

Embodiments of the present invention will be described in detail hereinafter. In the present specification, “to” that indicates a numerical range encompasses that the numerical values before and after “to” are included as the lower limit and the upper limit, respectively, unless otherwise specified.

### <Adhesive Electrode for Biosignal Acquisition>

The adhesive electrode for biosignal acquisition according to the present embodiment will be described. Note that the “bio” or “biological organism” means human bodies (humans), animals, such as cows, horses, pigs, chickens, dogs, or cats, or the like. The biosensor according to the present embodiment can be suitably used for biological organisms, particularly for human bodies. In the present embodiment, as one example, a case where the biological organism is a human will be described.

The adhesive electrode for biosignal acquisition according to the present embodiment is a bioelectrode, which is adhered to a part (e.g., skin, scalp, forehead, or the like) of a biological organism and detects biological information. In the present embodiment, a case where the adhesive electrode for biosignal acquisition is adhered to the skin of a human and acquires electrical signals (biosignals) associated with biological information of the human will be described. Note that the biosignals are, for example, electric signals representing electrocardiogram wave-forms, electroencephalograms, pulses, or the like.

The adhesive electrode for biosignal acquisition according to the present embodiment has a sheet shape. One side (one end side) of the adhesive electrode for biosignal acquisition in the longitudinal direction may be formed into a substantially rectangular shape in a plan view, and the other side (the other end side) in the longitudinal direction may be formed into a substantially arc shape in a plan view. The adhesive electrode for biosignal acquisition is, for example, brought into contact with and adhered to the skin, which is one example of a biological organism, to measure a potential difference (polarization voltage) between the skin and the adhesive electrode for biosignal acquisition so that the adhesive electrode for biosignal acquisition can be used to detect electrical signals (biosignals) associated with the biological information of a subject.

Note that the adhesive electrode for biosignal acquisition may have a shape other than the sheet shape, such as a rod shape or the like. Further, the shape of the adhesive electrode for biosignal acquisition in a plan view is not limited to the above shape, and may be designed to be any appropriate shape according to the intended use or the like, and may be formed into any shape, such as a substantially rectangular shape, a substantially polygonal shape, a substantially circular shape, a substantially elliptical shape, or the like.

A thickness of the adhesive electrode for biosignal acquisition according to the present embodiment may be any thickness within a range in which a strength, flexibility, low resistance, and conductivity can be assured, according to the intended use, a size of the adhesive electrode, or the like. Note that the thickness of the adhesive electrode for biosignal acquisition is a length of the adhesive electrode for biosignal acquisition in the direction vertical to the surface of the adhesive electrode for biosignal acquisition. The thickness of the adhesive electrode for biosignal acquisition is, for example, the thickness measured at an arbitrary area in a cross-section of the adhesive electrode for biosignal acquisition. In the case where the thickness is measured at two or more points in the arbitrary area, the thickness may be the arithmetic mean of the values of the thickness measured at the two or more points.

The adhesive electrode for biosignal acquisition according to the present embodiment is an electrode that exhibits adhesion (adhesive electrode). The adhesive electrode for biosignal acquisition includes a conductive polymer, an aqueous emulsion adhesive, and a nitrogen-containing additive, and may further include optional components, such as a moisturizing agent, an additive, and the like.

The present inventors have focused on the fact that, when the adhesive electrode for biosignal acquisition including the conductive polymer and the aqueous emulsion adhesive is used, the adhesive strength of the adhesive electrode for biosignal acquisition decreases due to the moisture absorbed in the aqueous emulsion adhesive as the adhesive electrode for biosignal acquisition absorbs moisture, such as sweat. The present inventors have found that, if the nitrogen-containing additive is added to the adhesive electrode for biosignal acquisition including the conductive polymer and the aqueous emulsion adhesive, the nitrogen-containing additive promptly absorbs moisture present in the surroundings of the adhesive electrode for biosignal acquisition, such as sweat at an interface between the adhesive electrode for biosignal acquisition and the skin, or the like, thereby minimizing reduction in adhesion of the adhesive electrode for biosignal acquisition. The present inventors have found that, since the adhesive electrode for biosignal acquisition includes the predetermined amount of the nitrogen-containing additive, water resistance is enhanced, the adhesive strength sufficient for the adhesive electrode for biosignal acquisition to be adhered to the skin is maintained, and the contact impedance with the skin is improved, so that the adhesive electrode for biosignal acquisition that can perform stable measurement with high sensitivity can be obtained.

As the conductive polymer included in the adhesive electrode for biosignal acquisition according to the present embodiment, for example, a polythiophene-based conductive polymer, a polyaniline-based conductive polymer, a polypyrrole-based conductive polymer, a polyacetylene-based conductive polymer, a polyphenylene-based conductive polymer, a derivative of any of the foregoing, a composite of any of the foregoing, or the like can be used. The above conductive polymers may be used alone or in combination. Among the above conductive polymers, a composite in which polythiophene is doped with polyaniline serving as a dopant is preferably used. Among the composites between the polythiophene and the polyaniline, PEDOT/PSS, in which poly(3,4-ethylenedioxythiophene) (PEDOT) is doped with polystyrene sulfonate (poly(4-styrenesulfonate) (PSS)), is more preferably used because a resultant adhesive electrode for biosignal acquisition has lower conduction impedance with a biological organism and high conductivity.

The aqueous emulsion adhesive included in the adhesive electrode for biosignal acquisition according to the present embodiment is used as a binder resin of the adhesive electrode for biosignal acquisition. The aqueous emulsion adhesive has a function of improving adhesion and flexibility of the adhesive electrode for biosignal acquisition. Since the aqueous emulsion adhesive is included in the adhesive electrode for biosignal acquisition, the adhesive electrode for biosignal acquisition has low elasticity, and the shape adaptability of the adhesive electrode for biosignal acquisition to conform to the surface texture of the skin is improved.

As the aqueous emulsion adhesive, an acrylic emulsion adhesive is preferably used.

As the acrylic emulsion adhesive, a silane-based emulsion adhesive including a water-dispersible copolymer and an organic liquid component compatible with the water-dispersible copolymer is preferably used.

The water-dispersible copolymer is a polymer obtained by copolymerizing a monomer mixture including alkyl (meth)acrylate with a silane-based monomer that is copolymerizable with the alkyl (meth)acrylate.

The monomer mixture including alkyl (meth)acrylate is a monomer mixture including alkyl (meth)acrylate as a main component, preferably in an amount of 50 wt % to 100 wt %.

As the alkyl (meth)acrylate, a linear or branched alkyl ester, in which an alkyl group has a carbon number of 1 to 15, and preferably 1 to 9, is used. Specific examples of the alkyl (meth)acrylate include alkyl (meth)acrylate including a linear or branched alkyl group, such as methyl (meth)acrylate, ethyl (meth)acrylate, n-butyl (meth)acrylate, isobutyl (meth)acrylate, pentyl (meth)acrylate, hexyl (meth)acrylate, heptyl (meth)acrylate, n-octyl (meth)acrylate, isooctyl (meth)acrylate, 2-ethylhexyl (meth)acrylate, n-nonyl (meth)acrylate, isononyl (meth)acrylate, decyl (meth)acrylate, undecyl (meth)acrylate, tridecyl (meth)acrylate, and the like. The above examples of the alkyl (meth)acrylate may be used alone or in combination.

The monomer mixture including alkyl (meth)acrylate may include a carboxyl group-containing monomer that is copolymerizable with the alkyl (meth)acrylate.

The carboxyl group-containing monomer that is copolymerizable with the alkyl (meth)acrylate is a polymerizable compound including a carboxyl group in a molecular structure of the polymerizable compound, and is not particularly limited as long as the carboxyl group-containing monomer is copolymerizable with the alkyl (meth)acrylate. Examples of the carboxyl group-containing monomer that is copolymerizable with the alkyl (meth)acrylate include (meth)acrylic acid, itaconic acid, maleic acid, maleic anhydride, 2-methacryloyloxyethyl succinic acid, and the like. In particular, acrylic acid is preferred.

In view of hydrolysis of the silane-based monomer or adjustment of adhesion to be obtained, the carboxyl group-containing monomer is preferably included in an amount of 0.1 wt % to 10 wt % relative to 100 wt % of the monomer mixture including alkyl (meth)acrylate.

The silane-based monomer copolymerizable with the alkyl (meth)acrylate is not particularly limited as long as the silane-based monomer copolymerizable with the alkyl (meth)acrylate is a polymerizable compound including a silicon atom, and is copolymerizable with the alkyl (meth)acrylate. In view of excellent comopolymerizability with the alkyl (meth)acrylate, the silane-based monomer copolymerizable with the alkyl (meth)acrylate is preferably a silane compound having a (meth)acryloyl group, such as a (meth)acryloyloxy alkyl silane derivative, or the like, is preferred. Examples of the silane-based monomer include 3-(meth)acryloyloxypropyltrimethoxysilane, 3-(meth)acryloyloxypropyltriethoxysilane, 3-(meth)acryloyloxypropylmethyldimethoxysilane, 3-(meth)acryloyloxypropylmethyldiethoxysilane, and the like. The above silane-based monomers may be used alone or in combination.

Further, as a silane-based monomer other than the above silane-based monomers, for example, vinyltrimethoxysilane, vinyltriethoxysilane, 4-vinylbutyltrimethoxysilane, 4-vinylbutyltriethoxysilane, 8-vinyloctyltrimethoxysilane, 8-vinyloctyltriethoxysilane, 10-methacryloyloxydecyltrimethoxysilane, 10-acryloyloxydecyltrimethoxysilane, 10-methacryloyloxydecyltriethoxysilane, 10-acryloyloxydecyltriethoxysilane, or the like can be used.

The silane-based monomer is preferably copolymerized with the monomer mixture including alkyl (meth)acrylate in an amount of 0.005 wt % to 2 wt % relative to 100 wt % of the monomer mixture including alkyl (meth)acrylate.

The silane-based monomer is copolymerized with the monomer mixture including alkyl (meth)acrylate so that the silane compound, which will become a crosslinking point, can be homogeneously distributed within a molecule of a resultant copolymer. Thus, the aqueous emulsion adhesive has excellent cohesive strength in spite of being water dispersible because the inner portion and outer portion of each particle of the aqueous emulsion adhesive are uniformly crosslinked, and has excellent fixability and sweat resistant fixability in addition to low skin irritability owing to the addition of the organic liquid component.

The water-dispersible copolymer may be obtained by optionally copolymerizing with a monomer copolymerizable with the alkyl (meth)acrylate other than the above silane-based monomer and the carboxyl group-containing monomer, as necessary. The monomer copolymerizable with the alkyl (meth)acrylate other than the silane-based monomer and the carboxyl group-containing monomer can be used for the purpose of adjusting the cohesive force of the adhesive electrode for biosignal acquisition when the aqueous emulsion adhesive is formed into a sheet shape or the like, improving compatibility with the organic liquid component, or the like. The amount of the monomer copolymerizable with the alkyl (meth)acrylate other than the silane-based monomer and the carboxyl group-containing monomer can be appropriately set according to the intended purpose by replacing a part of the amount of the alkyl (meth)acrylate with the monomer copolymerizable with the alkyl (meth)acrylate other than the silane-based monomer and the carboxyl group-containing monomer.

Examples of the monomer copolymerizable with the alkyl (meth)acrylate other than the silane-based monomer and the carboxyl group-containing monomer include: sulfo group-containing monomers, such as styrenesulfonic acid, allyl sulfonic acid, sulfopropyl (meth)acrylate, (meth)acryloyloxynaphthalenesulfonic acid, acrylamidemethylpropanesulfonic acid, and the like; hydroxyl group-containing monomers, such as hydroxyethyl (meth)acrylate, hydroxypropyl (meth)acrylate, and the like; amide group-containing monomers, such as (meth)acrylamide, dimethyl (meth)acrylamide, N-butylacrylamide, N-methylol (meth)acrylamide, N-methylolpropane (meth)acrylamide, and the like; alkylaminoalkyl (meth)acrylate, such as aminoethyl (meth)acrylate, dimethylaminoethyl (meth)acrylate, tert-butylaminoethyl (meth)acrylate, and the like; alkoxyalkyl (meth)acrylate, such as methoxyethyl (meth)acrylate, ethoxyethyl (meth)acrylate, and the like; alkoxy group-containing (or ether bond at a side chain) (meth)acrylate, such as methoxy ethylene glycol (meth)acrylate, tetrahydrofurfuryl (meth)acrylate, methoxyethylene glycol (meth)acrylate, methoxydiethylene glycol (meth)acrylate, methoxy polyethylene glycol (meth)acrylate, methoxy polypropylene glycol (meth)acrylate, and the like; and vinyl-based monomers, such as (meth)acrylonitrile, vinyl acetate, vinyl propionate, N-vinyl-2-pyrrolidone, methylvinylpyrrolidone, vinylpyridine, vinylpiperidine, vinylpyrimidine, vinylpiperazine, vinylpyrazine, vinylpyrrole, vinylimidazole, vinylcaprolactam, vinyloxazole, vinylmorpholine, and the like. The above monomers may be used alone or in combination.

The water-dispersible polymer can be prepared as an aqueous dispersion of an alkyl (meth)acrylate copolymer, for example, by subjecting a mixture that includes the monomer mixture including alkyl (meth)acrylate and the silane-based monomer to typical emulsification polymerization.

As the polymerization method, general batch polymerization, continuous dripping polymerization, divided dripping polymerization, or the like can be employed. The polymerization temperature is, for example, from 20° C. to 100° C.

A polymerization initiator used for the polymerization is not particularly limited, and a typical component used as a polymerization initiator can be used.

A chain transfer agent may be used in the polymerization to adjust the degree of polymerization. The chain transfer agent is not particularly limited, and a typical component used as a chain transfer agent may be used.

Other than the method described above, the water-dispersible copolymer may be prepared by forming a copolymer of the monomer mixture including (meth)acrylate and the silane-based monomer according to a method other than emulsification polymerization, followed by dispersing the copolymer in water using an emulsifier.

Since the organic liquid component included in the acrylic emulsion adhesive is blended with the water-dispersible copolymer, excellent adhesion to the surface of the skin is maintained, damages to keratin at the time of peeling from the surface of the skin can be reduced, and pain at the time of peeling can be also reduced.

The organic liquid component is preferably liquid at ambient temperature, and preferably has good compatibility with the water-dispersible copolymer. Note that “compatibility” refers to a state in which the organic liquid component is homogeneously dissolved and incorporated in the water-dispersible copolymer, and cannot be visually recognized.

Examples of the organic liquid component include an ester between a monobasic acid or polybasic acid having a carbon number of 8 to 18 and a branched alcohol having a carbon number of 14 to 18, an ester between an unsaturated fatty acid or branched acid having a carbon number of 14 to 18 and a tetrahydric or lower alcohol, and the like.

Examples of the ester between the monobasic acid or polybasic acid having the carbon number of 8 to 18 and the branched alcohol having the carbon number of 14 to 18 include isostearyl laurate, isocetyl myristate, octyldodecyl myristate, isostearyl palmitate, isocetyl stearate, octyldodecyl oleate, diisostearyl adipate, diisocetyl sebacate, trioleyl trimellitate, triisocetyl trimellitate, and the like.

Examples of the unsaturated fatty acid or branched acid having the carbon number of 14 to 18 include myristoleic acid, oleic acid, linoleic acid, linolenic acid, isopalmitic acid, isostearic acid, and the like.

Examples of the tetrahydric or lower alcohol include ethylene glycol, propylene glycol, glycerin, trimethylol propane, pentaerythritol, sorbitan, and the like.

An amount of the organic liquid component can be appropriately set according to the water-dispersible copolymer and the organic liquid component for use, or the like. For example, the amount of the organic liquid component may be from 20 wt % to 80 wt % relative to 100 wt % of the water-dispersible copolymer.

In the case where the acrylic emulsion adhesive is a silane-based emulsion adhesive, specifically, a silane-based emulsion adhesive including 2-ethylhexyl acrylate, methyl methacrylate, acrylic acid, and 3-methacryloxypropyltrimethoxysilane can be used as the acrylic emulsion adhesive.

Further, as the acrylic emulsion adhesive, a two-component or three-component acrylic emulsion adhesive including the monomer mixture including alkyl (meth)acrylate, and the carboxyl group-containing monomer can be used. The above acrylic emulsion adhesives may include a solvent or other components in appropriate amounts with which performance of the solvent or other components can be ensured.

The monomer mixture including alkyl (meth)acrylate, which is included in the two-component or three-component acrylic emulsion adhesive, is the same as the alkyl (meth)acrylate included in the above silane-based emulsion adhesive, and therefore the details will be omitted.

The carboxyl group-containing monomer is preferably a carboxyl group-containing monomer copolymerizable with alkyl (meth)acrylate. The carboxyl group-containing monomer copolymerizable with the alkyl is the same as the carboxyl group-containing monomer included in the above monomer mixture including (meth)acrylate, and therefore the details will be omitted.

As the two-component acrylic emulsion adhesive, specifically, an adhesive that includes 2-ethylhexylacrylate serving as the monomer mixture including alkyl (meth)acrylate, and acrylic acid serving as the carboxyl group-containing monomer mixture can be used.

As the three-component acrylic emulsion adhesive, specifically, an adhesive that includes 2-ethylhexylacrylate and methylacrylate serving as the monomer mixture including alkyl (meth)acrylate, and acrylic acid serving as the carboxyl group-containing monomer mixture can be used.

The average particle size of the aqueous emulsion adhesive is preferably from 100 nm to 1.0 μm, more preferably from 100 nm to 500 nm, and yet more preferably from 100 nm to 300 nm. When the average particle size of the aqueous emulsion adhesive is within the above preferred range, adhesive strength and water resistance can be imparted to the adhesive electrode for biosignal acquisition.

The shape of the aqueous emulsion adhesive is not particularly limited, and may be, for example, a spherical shape, an ellipsoidal shape, a spindle shape, a fragment shape, a plate shape, a columnar shape, or the like.

The average particle size is a volume-based mean particle diameter of effective diameters. For example, the average particle size is a particle diameter (median diameter) at which a cumulative amount of the particles accumulated from the smaller particles is 50% on a volume basis on the particle size distribution curve of the -based emulsion adhesive or acrylic emulsion adhesive measured by a laser diffraction/scattering method, a dynamic light scattering method, or the like.

An amount of the aqueous emulsion adhesive is preferably from 35 wt % to 90 wt %, more preferably from 40 wt % to 85 wt %, and yet more preferably from 50 wt % to 80 wt %, relative to 100 wt % of the adhesive electrode for biosignal acquisition. When the amount of the aqueous emulsion adhesive is within the preferred range, an adhesive strength and flexibility are imparted to the adhesive electrode for biosignal acquisition, and reduction in conductivity can be minimized.

The nitrogen-containing additive included in the adhesive electrode for biosignal acquisition according to the present embodiment has characteristics such that the nitrogen-containing additive absorbs moisture that enters the adhesive electrode for biosignal acquisition or comes into contact with the surface of the adhesive electrode for biosignal acquisition 1. Even if the aqueous emulsion adhesive included in the adhesive electrode for biosignal acquisition has a characteristic of water absorption, the nitrogen-containing additive absorbs the external moisture entering the adhesive electrode for biosignal acquisition or coming into contact with the adhesive electrode for biosignal acquisition, and therefore the adhesion of the aqueous emulsion adhesive can be maintained. In the case where the conductive polymer is, for example, PEDOT-PSS, moreover, PSS also exhibits water absorption, and has a characteristic such that PSS readily absorbs the moisture. Since the nitrogen-containing additive absorbs the moisture, the PSS can maintain a function as a dopant of the PEDOT and a dispersing agent so that high conductivity of the adhesive electrode for biosignal acquisition can be maintained.

The nitrogen-containing additive includes a nitrogen-containing compound that includes nitrogen atoms, a nitrogen-containing resin (a resin including nitrogen atoms), or the like. The nitrogen-containing additive may be composed only of a nitrogen-containing compound or a nitrogen-containing resin.

As the nitrogen-containing compound, an imidazole compound, low-molecular-weight amines, polyvinylpyrrolidone, polyethyleneimine, peptide, amino acid, melamine, or the like is used. Among the above-listed compounds, an imidazole compound is preferred in view of water absorption and flexibility. The above-listed compounds may be used alone or in combination.

The imidazole compound is an organic structure including an imidazole group. Examples of the imidazole compound include heterocyclic amines.

Examples of the heterocyclic amines include imidazole, 2-methylimidazole, 2-propylimidazole, 2-undecylimidazole, 2-phenylimidazole, N-methylimidazole, 1-(2-hydroxyethyl) imidazole, 2-ethyl-4-methylimidazole, 1,2-dimethylimidazole, 1-benzyl-2-methylimidazole, 1-benzyl-2-phenylimidazole, 1-cyanoethyl-2-methylimidazole, 1-cyanoethyl-2-ethyl-4-methylimidazole, 2-phenyl-4,5-dihydroxymethylimidazole, 1-acetylimidazole, 4,5-imidazoledicarboxylic acid, dimethyl 4,5-imidazoledicarboxylate, benzimidazole, 2-aminobenzimidazole, 2-aminobenzimidazole-2-sulfonic acid, 2-amino-1-methylbenzimidazole, 2-hydroxybenzimidazole, 2-(2-pyridyl)benzimidazole, and the like. Among the above-listed heterocyclic amines, imidazole is preferred. The above heterocyclic amines may be used alone or in combination.

Moreover, the imidazole group of the imidazole compound functions as a neutralizing agent, for example, in the range of pH 3.5 to pH 6.5, and therefore the imidazole compound can neutralize the pH of the adhesive electrode for biosignal acquisition.

Examples of the low-molecular-weight amines include ethylenediamine, ethylamine, methylamine, dimethylamine, trimethylamine, triethylamine, triethanolamine, monoethanolamine, 2-dimethylaminoethanol, diethylamine, diethylaminoethanol, diisopropanolamine, aniline, benzylamine, pyridine, pyrazole compounds, and the like. The above-listed low-molecular-weight amines may be used alone or in combination.

The nitrogen-containing resin is not particularly limited as long as the nitrogen-containing resin is a nitrogen-containing resin that exhibits water absorption. For example, a urea resin or the like can be used.

An amount of the nitrogen-containing additive is from 0.01 wt % to 3.0 wt %, preferably from 0.1 wt % to 2.5 wt %, more preferably from 0.3 wt % to 2 wt %, and yet more preferably from 0.5 wt % to 2.0 wt %, relative to a total amount (100 wt %) of the adhesive electrode for biosignal acquisition.

An amount of nitrogen in the nitrogen-containing additive is preferably from 5 wt % to 45 wt %, more preferably from 8 wt % to 40 wt %, and yet more preferably from 9 wt % to 30 wt %, relative to a total amount (100 wt %) of the nitrogen-containing additive.

In the case where a mixture of two or more nitrogen-containing additives is used, a blending ratio of the nitrogen-containing additives is not particularly limited, and may be set to an appropriate ratio according to kinds of the nitrogen-containing additives or the like. For example, the blending ratio is preferably from 10:1 to 1:10, more preferably from 5:1 to 1:5, and yet more preferably from 3:1 to 1:3.

As described above, the adhesive electrode for biosignal acquisition may include, as optional components, a moisturizing agent, an additive, or the like.

The moisturizing agent has a function of improving conductivity of the adhesive electrode for biosignal acquisition, and improving adhesive strength and flexibility.

Examples of the moisturizing agent include: polyol compounds, such as glycerin, ethylene glycol, propylene glycol, sorbitol, polymers of the foregoing, and the like; and aprotonic compounds, such as N-methylpyrrolidone (NMP), dimethylformaldehyde (DMF), N—N′-dimethylacetoamide (DMAc), dimethylsulfoxide (DMSO), and the like. The above moisturizing agents may be alone or in combination. Among the above moisturizing agents, glycerin is preferred in view of compatibility with other components.

An amount of the moisturizing agent is preferably from 2 wt % to 60 wt %, more preferably from 3 wt % to 50 wt %, and yet more preferably from 5 wt % to 35 wt %, relative to 100 wt % of the adhesive electrode for biosignal acquisition. When the amount of the moisturizing agent is within the above preferred range, adhesive strength of the adhesive electrode for biosignal acquisition can be improved and high adhesion to the skin surface can be maintained. In addition, an elastic storage modulus can be reduced, and viscoelasticity can be increased. Thus, noise generated during use can be significantly reduced. Further, the adhesive electrode for biosignal acquisition can minimize absorption of external water, thereby inhibiting swelling.

The additive exhibits a neutralization effect against the conductive polymer so that the conductive polymer is neutralized, and also has a function of improving flexibility. In the case where the conductive polymer is, for example, PEDOT-PSS, the additive neutralizes PSS, which is a strong acid, to form an organic salt, thereby improving water absorption and exhibiting an effect of insolubilizing the organic salt in water.

As the additive, for example, an imidazole compound or the like is preferably exemplified. The imidazole compound is the same as the above-described imidazole compound, and therefore the details will be omitted.

An amount of the additive is preferably from 0.5 wt % to 2.4 wt %, more preferably from 0.7 wt % to 2.2 wt %, and yet more preferably from 0.8 wt % to 2.0 wt %, relative to 100 wt % of the adhesive electrode for biosignal acquisition.

A production method for the adhesive electrode for biosignal acquisition is not particularly limited. One example of the production method for the adhesive electrode for biosignal acquisition will be described. For example, first, a conductive polymer and water are mixed to prepare a conductive polymer-containing solution (conductive polymer-containing solution preparation step).

Mixing conditions, such as a blending ratio between the conductive polymer and water, a mixing time, and the like, as well as a temperature of the conductive polymer-containing solution or the like, are not particularly limited, and may be appropriately set to arbitrary values as long as the conductive polymer can be sufficiently mixed in the conductive polymer-containing solution.

Next, an aqueous emulsion adhesive, a moisturizing agent, and a nitrogen-containing additive are mixed in the conductive polymer-containing solution to prepare an adhesive-composition aqueous solution (mixing step). The adhesive-composition aqueous solution is used as an adhesive-electrode forming composition.

Mixing conditions, such as a blending ratio between the conductive polymer-containing solution, the aqueous emulsion adhesive, the moisturizing agent, and the nitrogen-containing additive, a mixing time, and the like, as well as a temperature of the adhesive-composition aqueous solution or the like, are not particularly limited, and may be appropriately set to arbitrary values as long as the aqueous emulsion adhesive, the moisturizing agent, and the nitrogen-containing additive are sufficiently mixed in the conductive polymer-containing solution.

Next, after applying the adhesive-composition aqueous solution to a surface (coating surface) of a release backing material, the applied adhesive-composition aqueous solution is dried to evaporate the moisture in the adhesive-composition aqueous solution (coating and drying step). Since the aqueous emulsion adhesive is in the form of particles, as the adhesive-composition aqueous solution is applied to the coating surface of the release backing material, particles of the aqueous emulsion adhesive are bonded and fused to one another, thereby forming a coating film, which is a cured product of the adhesive-electrode forming composition.

As the release backing material, a release liner, a core material, or the like can be used. As the release liner, a resin film, such as a polyethylene terephthalate (PET) film, a polyethylene (PE) film, a polypropylene (PP) film, a polyamide (PA) film, a polyimide (PI) film, a fluororesin film, or the like can be used. As the core material, a resin film, such as a PET film, a PI film, or the like; a ceramic sheet; a metal film, such as aluminum foil or the like; a resin film reinforced with glass fibers, a plastic non-woven fiber, or the like; or a silicone substrate or a glass substrate can be used.

A method of applying the adhesive-composition aqueous solution to the coating surface of the release backing material is not particularly limited as long as the adhesive-composition aqueous solution can be applied to the release backing material. A general coating method may be used.

As the coating method, a method using roll coating, screen coating, gravure coating, spin coating, reverse coating, bar coating, blade coating, spray coating, air knife coating, dipping, dispensing, or the like, a method of dripping a small amount of the adhesive-composition aqueous solution onto the coating surface of the base and spreading the adhesive-composition aqueous solution with a doctor blade, or the like can be used. The adhesive-composition aqueous solution is uniformly applied onto the coating surface according to any of the above coating methods.

Drying conditions for the adhesive-composition aqueous solution applied to the coating surface of the release backing material are not particularly limited as long as the drying conditions are conditions under which the adhesive-composition aqueous solution applied to the release backing material can be dried. General drying conditions may be used.

At the time of drying, drying may be performed at room temperature, or heating may be performed using a dryer. As the dryer, a general dryer, such as a drying oven, a vacuum oven, an air circulation oven, a hot air dryer, a far infrared dryer, a microwave decompression dryer, a high-frequency dryer, or the like can be used. The adhesive-composition aqueous solution applied to the coating surface of the base may be dried using any of the above dryers according to a method where the internal atmosphere of the dryer is heated at a high temperature, a method where the base is heated, a method where hot air is blown onto the adhesive-composition aqueous solution, a method where the adhesive-composition aqueous solution is irradiated with infrared rays, microwaves, high frequency waves, or the like.

A heating temperature and a heating time when the adhesive-composition aqueous solution is dried using the dryer are a temperature and a time by which the moisture in the adhesive-composition aqueous solution can be evaporated. The heating temperature may be, for example, from 100° C. to 200° C. In the case where a crosslinking agent is included in the conductive composition, evaporation of the moisture in the adhesive-composition aqueous solution can be facilitated as long as the heating temperature is in the range of 100° C. to 200° C. The heating time of the adhesive-composition aqueous solution may be, for example, from 0.5 minutes to 300 minutes. When the heating time is from 0.5 minutes to 300 minutes, the moisture in the adhesive-composition aqueous solution can be sufficiently evaporated.

Next, the obtained cured product is punched (pressed) by a press machine or the like, as necessary, so that one or more through holes are formed in the surface of the cured product and the outer shape of the cured product can be formed into a predetermined shape (shaping step). Thus, an adhesive electrode for biosignal acquisition, which is a shaped product having one or more through holes 11 in the surface of the shaped product, and having a predetermined outer shape, is obtained.

Note that the cured product may be shaped by a laser processing machine instead of the press machine. In addition, the obtained cured product may have only the one or more through holes 11 formed in the surface of the cured product, or may have only the outer shape formed into the predetermined shape. In the case where the cured product can be used as an adhesive electrode for biosignal acquisition as it is, the cured product may be used as the adhesive electrode for biosignal acquisition without being shaped or the like.

As described above, the adhesive electrode for biosignal acquisition according to the present embodiment includes the conductive polymer, the aqueous emulsion adhesive, and the nitrogen-containing additive, and an amount of the nitrogen-containing additive is from 0.01 wt % to 3.0 wt % relative to 100 wt % of the adhesive electrode for biosignal acquisition. Since the adhesive electrode for biosignal acquisition includes the nitrogen-containing additive in the amount of the above range, the nitrogen-containing additive can absorb the external moisture entering the adhesive electrode for biosignal acquisition 1, such as sweat on the skin, the external moisture coming into contact with the adhesive electrode for biosignal acquisition, or the like, thereby enhancing water resistance. Thus, the adhesive electrode for biosignal acquisition can minimize the reduction in the performance of the aqueous emulsion adhesive so that the adhesive electrode for biosignal acquisition can strongly adhere to the skin. Even in the state in which the adhesive electrode for biosignal acquisition includes the moisture, therefore, adhesion substantially equivalent to the adhesion before moisture absorption (dry state) can be maintained.

Further, the adhesive electrode for biosignal acquisition can maintain high conductivity because the dielectric constant is reduced by water absorption, and the moisture can fill the gap formed at the interface between the adhesive electrode for biosignal acquisition and the skin.

Therefore, the adhesive electrode for biosignal acquisition can improve contact impedance and has excellent adhesion even in the state in which external moisture, such as sweat generated from the skin, is absorbed and included in the adhesive electrode for biosignal acquisition during use. Thus, the adhesive electrode for biosignal acquisition can perform stable measurement with high sensitivity.

In general, as adhesion of an adhesive electrode decreases, contact impedance with respect to the skin decreases, and the electrode may be shifted away or peeled off if the skin surface is deformed even slightly due to the body movement, thereby making stable measurement of biosignals difficult. In the present embodiment, the adhesive electrode for biosignal acquisition can maintain adhesive strength even when the moisture derived from sweat on the interface with the skin or the like is absorbed. Thus, the adhered state of the adhesive electrode for biosignal acquisition to the skin can be maintained even when the skin surface is deformed due to the body movement, and the contact impedance can be maintained low.

The adhesive electrode for biosignal acquisition can inhibit the increase in the conduction impedance, and can maintain the contact impedance at a predetermined value (e.g., 50 kΩ) or lower even after immersing the adhesive electrode for biosignal acquisition in water for a predetermined time (e.g., 60 seconds).

In the case where PEDOT/PSS is used as the conductive polymer, PSS, which is used as a dopant in the PEDOT/PSS, exhibits a high level of water absorption and has a characteristic such that PSS readily absorbs water. Since the adhesive electrode for biosignal acquisition includes the nitrogen-containing additive, the moisture present in the surroundings of the adhesive electrode for biosignal acquisition, such as sweat generated from the skin, or the like, is absorbed by the nitrogen-containing additive so that PSS can maintain a function as a dispersing agent. Further, PSS also functions as a dispersing agent, and the PEDOT/PSS can be included in the adhesive electrode for biosignal acquisition in the dispersed state. Therefore, the adhesive electrode for biosignal acquisition can maintain conductivity even when PEDOT/PSS is used as the conductive polymer and the moisture is included during use.

Since the adhesive electrode for biosignal acquisition includes the conductive polymer and the aqueous emulsion adhesive, low resistance is retained, the conductivity is exhibited, the adhesive strength against the skin is ensured, and flexibility is imparted, thereby improving shape adaptability to conform to the surface of the skin.

Note that a measurement method of the contact impedance of the adhesive electrode for biosignal acquisition is not particularly limited, and the contact impedance can be measured by any appropriate method. For example, an electrode cut to have an area of a predetermined size (e.g., 4 cm²) is prepared as a test piece, an end of a copper foil tape having a predetermined size (e.g., 5 mm in width×10 cm in length) is bonded to the end of the electrode, and the opposite surface of the copper foil tape to the surface bonded to the electrode is secured to a cable having clips. The clip on the opposite side of the cable is coupled to an impedance analyzer, the electrode is adhered to a part of the skin of a subject, and the contact impedance with the skin is measured for a predetermined time (e.g., 1 minute). Note that as the measurement method of the contact impedance of the adhesive electrode for biosignal acquisition 1, a biosensor in which an electrode is arranged on an adhering surface is produced so that the electrode and the skin can be in contact with each other, instead of the test piece of the electrode, and the contact impedance may be measured using the biosensor.

A measurement method of the adhesive strength of the adhesive electrode for biosignal acquisition is not particularly limited, and the adhesive strength can be measured by any appropriate method. For example, the adhesive strength of the adhesive electrode for biosignal acquisition may be determined by performing a 180° peel test to measure 180° peel strength. An electrode sheet cut to have an area (e.g., 4 cm²) of a predetermined size is prepared as a test piece, the electrode sheet is bonded to and backed by a support substrate (e.g., a PET film) to produce a measuring sample. The measuring sample is cut into a predetermined size (e.g., 5 cm×1 cm), and is bonded to an adherend (stainless steel plate), followed by being processed in an autoclave. Thereafter, the load applied when the measurement sample is peeled off from the adherend is measured by a tensile tester under peeling conditions, i.e., a peeling angle of 180° and a predetermined peeling speed (e.g., 300 mm/min), to determine release strength (unit: N/10 mm). In the case where the electrode sheet is subjected to a water absorption process to absorb water, the same procedure is performed to measure the adhesive strength.

When the contact impedance and adhesive strength of the adhesive electrode for biosignal acquisition are measured, in addition to the case where the adhesive electrode for biosignal acquisition is in a dried state, the adhesive electrode for biosignal acquisition is subjected to a water absorption process, and the adhesive electrode for biosignal acquisition having a predetermined water absorption rate may be used. The water absorption process may be any process as long as water is absorbed in the adhesive electrode for biosignal acquisition. For example, the water absorption rate of the adhesive electrode for biosignal acquisition may be adjusted by exposing the adhesive electrode for biosignal acquisition to a recycled cloth or paper including water for a predetermined time. The water absorption rate of the adhesive electrode for biosignal acquisition may be measured based on the change in mass of the adhesive electrode for biosignal acquisition before and after exposing the adhesive electrode for biosignal acquisition to the recycled cloth or paper. For example, in the case where the mass of the adhesive electrode for biosignal acquisition exposed to the recycled cloth or paper for 1 second is increased by approximately 10 percent by mass, and the mass of the adhesive electrode for biosignal acquisition exposed to the recycled cloth or paper for 3 seconds is increased by approximately 30 percent by mass, the water absorption rates of the adhesive electrode for biosignal acquisition can be determined as 10 percent by mass and 30 percent by mass, respectively. The water absorption rate of the adhesive electrode for biosignal acquisition is increased from 10 percent by mass to 100 percent by mass based on the change in mass of the adhesive electrode for biosignal acquisition before and after water absorption, thereby producing the adhesive electrode for biosignal acquisition having a predetermined absorption. Note that a cut piece of the electrode sheet may be used as the adhesive electrode for biosignal acquisition.

The resistance of the adhesive electrode for biosignal acquisition can be evaluated by measuring sheet resistance (unit: Q/unit area) of the adhesive electrode for biosignal acquisition. The sheet resistance is surface resistance of the adhesive electrode for biosignal acquisition. The sheet resistance can be measured by a general method of measuring resistance. For example, the sheet resistance can be measured by an eddy current measurement method using a non-contact resistance measuring machine in accordance with JIS Z 2316-1:2014. The measurement range may be a predetermined range of a main surface of the adhesive electrode for biosignal acquisition.

In the adhesive electrode for biosignal acquisition, an amount of nitrogen in the nitrogen-containing additive can be from 9 wt % to 45 wt % relative to a total amount of the nitrogen-containing additive. Thus, the external moisture entering the adhesive electrode for biosignal acquisition can be assuredly absorbed by the nitrogen-containing additive, and therefore water resistance is assuredly enhanced and adhesion is stably maintained. Thus, the adhesive electrode for biosignal acquisition can improve contact impedance and has excellent adhesion even in a state in which the external moisture is absorbed and the moisture is contained in the adhesive electrode for biosignal acquisition.

In the adhesive electrode for biosignal acquisition, as the nitrogen-containing additive, at least one component selected from the group consisting of an imidazole compound, low-molecular-weight amines, and polyvinylpyrrolidone can be used. Thus, the external moisture entering the adhesive electrode for biosignal acquisition can be assuredly absorbed by the nitrogen-containing additive, water resistance can be assuredly enhanced, and adhesion is stably maintained. Thus, the adhesive electrode for biosignal acquisition can improve contact impedance and has excellent adhesion even in a state in which the external moisture is absorbed and the moisture is included in the adhesive electrode for biosignal acquisition.

In particular, since the adhesive electrode for biosignal acquisition includes an imidazole compound as the nitrogen-containing additive, flexibility is imparted and neutralization can be achieved, and therefore skin irritability is assuredly reduced, and flexibility can be enhanced.

In the adhesive electrode for biosignal acquisition, an acrylic emulsion adhesive can be used as the aqueous emulsion adhesive. Thus, the adhesive electrode for biosignal acquisition can minimize the reduction in adhesive strength while retaining the resistance, and shape adaptability to conform to the skin surface can be assuredly enhanced. Therefore, the adhesive electrode for biosignal acquisition can have high adhesive strength and shape adaptability to conform to the skin surface.

In the adhesive electrode for biosignal acquisition, as the acrylic emulsion adhesive, a silane-based emulsion adhesive including a water-dispersible copolymer and an organic liquid component can be used. Thus, the adhesive electrode for biosignal acquisition can assuredly maintain the viscoelasticity low so that the adhesive strength is enhanced and the shape adaptability to follow the skin surface can be further improved. Therefore, the adhesive electrode for biosignal acquisition can further enhance flexibility, and assuredly retain adhesion.

Further, the adhesive electrode for biosignal acquisition includes glycerin as a moisturizing agent, and therefore the glycerin functions as a dispersing agent of PEDOT used as the conductive polymer, thereby enhancing dispersibility of the PEDOT. Furthermore, the glycerin enhances crystallinity of the PEDOT used as the conductive polymer, thereby enhancing conductivity. Therefore, the adhesive electrode for biosignal acquisition can stably enhance conductivity.

As described above, the adhesive electrode for biosignal acquisition according to the present embodiment can maintain low contact impedance, has excellent adhesion to the skin, and can perform stable measurement with high sensitivity even when the external moisture is absorbed during use. Thus, the adhesive electrode for biosignal acquisition can be effectively used as a biosensor, particularly, an adhesive electrode for a biosensor (bioelectrode), which needs to exhibit adhesion to the skin of a human, high flexibility, and safety to the skin.

The embodiment has been described above, but the above embodiment is merely provided as an example and shall not be construed as limiting the present invention. The above embodiment can be performed in various other forms, and various combinations, omissions, substitutions, modifications or the like can be made without departing from the spirit of the invention. These embodiments and modifications are included in the scope and spirit of the invention, and are included within the scope of the invention described in the claims and the equivalents of the invention.

### EXAMPLES

The embodiment will be more specifically explained through Examples and Comparative Examples hereinafter, but the embodiment is not limited to Examples and Comparative Examples.

### <Production of Electrode Sheet>

### Example 1

### (Production of Conductive Composition)

In a vessel, 1.0 part by mass of PEDOT/PSS pellets (“Orgacon DRY,” produced by AGFA Materials Japan, LTD.) serving as a conductive polymer and 23 g of water were added, and the resultant mixture was stirred by a stirring mixer (THINKY MIXER, THINKY CORPORATION) at the rotational speed of 2,000 rpm for 10 minutes. Subsequently, 12.0 g of an emulsion adhesive (2EHA/MMA/AA/=90/10/4, solid content of 52%) serving as a binder resin, 2.0 g of glycerin serving as a moisturizing agent, and 0.1 g of an imidazole aqueous solution having the imidazole concentration of 30% serving as a nitrogen-containing additive were added, the resultant mixture was mixed by stirring and defoaming using the stirring mixer at 2,200 rpm for 10 minutes, thereby preparing a homogeneous conductive-composition aqueous solution having a solid content of 24 wt %. The amount of the imidazole in the conductive-composition aqueous solution was 0.8 wt % relative to the total amount of the adhesive electrode for biosignal acquisition. Further, the amount of nitrogen in the nitrogen-containing additive was 41 wt % relative to the total amount of the nitrogen-containing additive.

### (Production of Electrode Sheet)

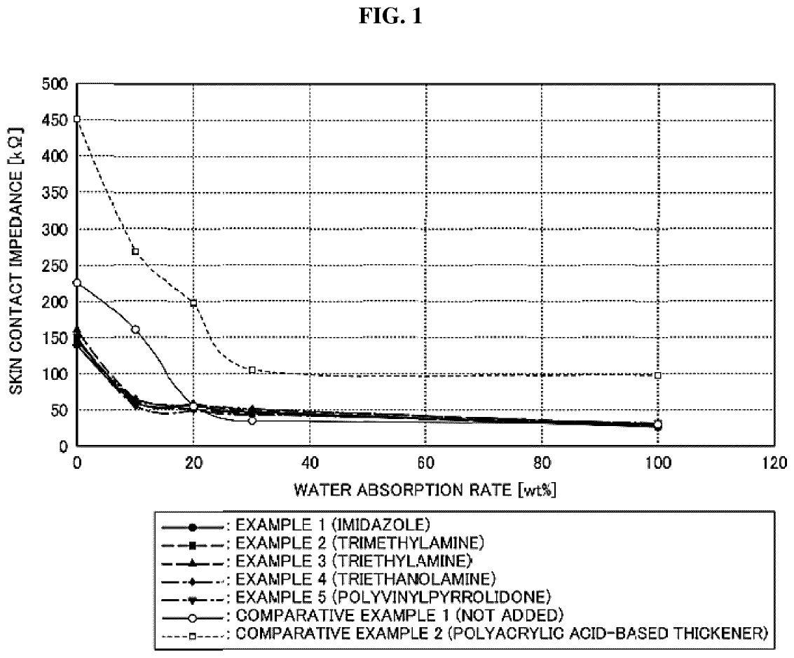

The prepared conductive-composition aqueous solution was applied onto a polyethylene terephthalate (PET) film (PET-50-SCA 1, produced by Fujiko Co., Ltd., thickness of 50 μm), which had been subjected to a surface treatment with a silicone-based release agent, using an applicator. Thereafter, the PET film, to which the conductive composition aqueous solution had been applied, was transported to a drying oven (SPHH-201, produced by ESPEC), and the conductive-composition aqueous solution was heated and dried at 130° C. for 3 minutes, thereby producing a cured product of the conductive composition. The cured product was punched (pressed) into a desired shape in a plan view as illustrated in FIG. 1 by punching (pressing) to form into a sheet shape, thereby producing an electrode sheet (bioelectrode) having a thickness of 30 μm.

### Examples 2 to 11

An electrode sheet was produced in the same manner as in Example 1, except that the nitrogen-containing additive used in the production of the conductive composition in Example 1 was changed to trimethylamine, triethylamine, triethanolamine, polyvinylpyrrolidone, or a mixture of imidazole and trimethylamine, and the amount of the foregoing was kept the same or changed as presented in Table 1.

### Comparative Example 1

An electrode sheet was produced in the same manner as in Example 1, except that the nitrogen-containing additive used in the production of the conductive composition in Example 1 was not used.

### Comparative Example 2

An electrode sheet was produced in the same manner as in Example 1, except that the nitrogen-containing additive used in the production of the conductive composition in Example 1 was changed to a polyacrylic acid-based thickener (A-10H, produced by TOAGOSEI CO., LTD.).

### Comparative Example 3

An electrode sheet was produced in the same manner as in Example 1, except that the nitrogen-containing additive used in the production of the conductive composition in Example 1 was changed to polyvinylpyrrolidone, and the amount of the nitrogen-containing additive was changed to 5 wt %.

The type of the nitrogen-containing additive included in the electrode sheet of each of Examples and Comparative Examples above is presented in Table 1.

### <Adjustment of Water Absorption Rate>

The electrode sheet produced in each of Examples and Comparative Examples was provided as multiple sheets, and the moisture was absorbed in part of the electrode sheets to have a predetermined water absorption rate (moisture absorption rate). The adjustment of the water absorption rate of the electrode sheet was performed by exposing the electrode sheet of each of Examples and Comparative Examples to a recycled cloth or paper including water for a predetermined time. The water absorption rate of the electrode sheet was calculated by determining the change in mass of the electrode sheet before and after being exposed to the recycled cloth or paper including water. Since the electrode sheet is increased by approximately 10 percent by mass when the electrode sheet is exposed to the recycled cloth or paper including water for 1 second, the water absorption rate for the above case is determined as 10 wt %. The electrode sheets were processed so that the water absorption rates of the electrode sheets were to be 10 percent by mass, 20 percent by mass, 30 percent by mass, 40 percent by mass, 50 percent by mass, and 100 percent by mass, respectively, based on the change in mass of the electrode sheets when being exposed to the recycled cloth or paper for a predetermined time, thereby producing samples of the electrode sheets having the predetermined water absorption rates.

### <Evaluations of Electrode Sheet>

The contact impedance, adhesive strength, and sheet resistance of the obtained electrode sheet of each of Examples and Comparative Examples were measured.

### [Measurement of Contact Impedance]

The electrode sheet was cut into pieces each having an area of 4 cm², and the electrode sheets having the water absorption rates of 0 percent by mass, 10 percent by mass, 20 percent by mass, 30 percent by mass, and 100 percent by mass, respectively, were prepared. An end of a copper foil tape cut into a width of 5 mm and a length of 10 cm was bonded to each of the prepared electrode sheets, and the opposite surface of the copper foil tape to the surface bonded to the electrode sheet was secured to a cable having alligator clips. The clip on the opposite side of the cable was coupled to an impedance analyzer (product name: IM3570, produced by HIOKI E. E. CORPORATION), followed by adhering the electrode sheet to the inner side of the arm of a subject, and contact impedance with the skin was measured for 1 minute. The measurement results of the contact impedance of the electrode sheets of each of Examples and Comparative Examples at corresponding water absorption rates are presented in Table 1. In addition, the relationship between the water absorption rate and the contact impedance of the electrode sheet of each of Examples and Comparative Example is depicted in FIG. 1.

### [Adhesive Strength]

The adhesive strength of the electrode sheet of each of Examples and Comparative Examples was evaluated by performing a 180° peel test to measure 180° peel strength. For the measurement of the adhesive strength, the electrode sheets having the water absorption rates of 0 percent by mass, 10 percent by mass, 20 percent by mass, 30 percent by mass, 40 percent by mass, 50 percent by mass, and 100 percent by mass, respectively, were used.

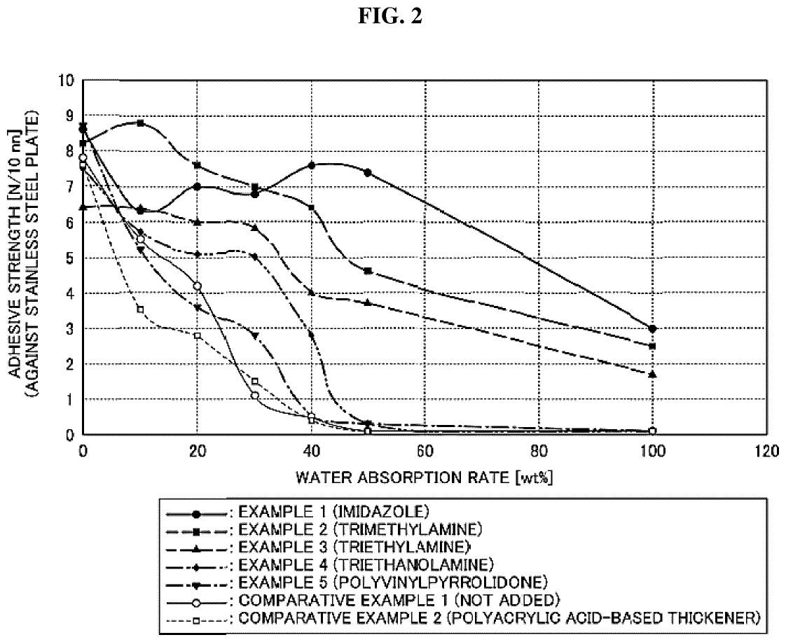

The electrode sheet of each of Examples and Comparative Examples, which had been cut to have an area of 4 cm², was prepared as a test piece of the electrode sheet. The electrode sheet was bonded to and backed by a PET film, thereby producing a measurement sample. The measurement sample was cut into the size of 5 cm×1 cm, and was bonded to a stainless steel plate (SUS304BA plate) serving as an adherend, followed by being processed in an autoclave. Thereafter, the load applied when the measurement sample was peeled off from the adherend was measured by a tensile tester (product name “Autograph AG-Xplus HS 6000 mm/min high-speed model (AG-50NX plus),” produced by Shimadzu Corporation), under peeling conditions, i.e., a peeling angle of 180° and peeling speed (tensile speed) of 300 mm/min, thereby determining peel strength (unit: N/10 mm). The measurement results of the adhesive strength of the electrode sheets of each of Examples and Comparative Examples at corresponding water absorption rates are presented in Table 2. In addition, the relationship between the water absorption rate and the adhesive strength of the electrode sheet of each of Examples and Comparative Examples is depicted in FIG. 2.

TABLE 1

| | Additive | | | | Contact impedance [kΩ] | | | | |
|---|---|---|---|---|---|---|---|---|---|
| | | | | Nitrogen | Water | Water | Water | Water | Water |
| | | | Amount | Amount | absorption | absorption | absorption | absorption | absorption |
| | Type | | [wt %] | [wt %] | rate: 0% | rate: 10% | rate: 20% | rate: 30% | rate: 100% |
| Ex. 1 | Nitrogen- | Imidazole | 0.8 | 41 | 140 | 61 | 55 | 47 | 27 |
| Ex. 2 | containing | Trimethylamine | 0.8 | 24 | 148 | 59 | 51 | 45 | 28 |
| Ex. 3 | additive | Triethylamine | 0.8 | 14 | 161 | 65 | 59 | 50 | 30 |
| Ex. 4 | | Triethanolamine | 0.8 | 9 | 146 | 63 | 57 | 51 | 29 |
| Ex. 5 | | Polyvinylpyrrolidone | 1.0 | 13 | 152 | 55 | 50 | 44 | 32 |
| Ex. 6 | | Imidazole | 0.4 | 41 | 145 | 64 | 61 | 54 | 33 |
| Ex. 7 | | Imidazole | 1.6 | 41 | 139 | 60 | 58 | 53 | 32 |
| Ex. 8 | | Imidazole | 2.4 | 41 | 157 | 69 | 65 | 61 | 37 |
| Ex. 9 | | Polyvinylpyrrolidone | 0.5 | 13 | 167 | 78 | 72 | 68 | 41 |
| Ex. 10 | | Polyvinylpyrrolidone | 2.0 | 13 | 153 | 60 | 58 | 57 | 40 |
| Ex. 11 | | Imidazole · | 0.8 · | 33 | 154 | 58 | 55 | 47 | 24 |
| | | Trimethylamine | 0.8 | | | | | | |
| Comp. | Not added | | 0.0 | 0 | 226 | 160 | 55 | 35 | 30 |
| Ex. 1 | | | | | | | | | |
| Comp. | Polyacrylic acid-based thickener | | 1.0 | 0 | 451 | 268 | 198 | 105 | 98 |
| Ex. 2 | | | | | | | | | |
| Comp. | Nitrogen- | Polyvinylpyrrolidone | 5.0 | 13 | 150 | 58 | 54 | 60 | 65 |
| Ex. 3 | containing | | | | | | | | |
| | additive | | | | | | | | |

TABLE 2

| | Additive | | | | Adhesive strength [N/10 mm] (against stainless steel plate) | | | | | | |
|---|---|---|---|---|---|---|---|---|---|---|---|
| | | | | Nitrogen | Water | Water | Water | Water | Water | Water | Water |
| | | | Amount | amount | absorption | absorption | absorption | absorption | absorption | absorption | absorption |
| | Type | | [wt %] | [wt %] | rate: 0% | rate: 10% | rate: 20% | rate: 30% | rate: 40% | rate: 50% | rate: 100% |
| Ex. 1 | Nitrogen- | Imidazole | 0.8 | 41 | 8.6 | 6.3 | 7.0 | 6.8 | 7.6 | 7.4 | 3.0 |
| Ex. 2 | containing | Trimethylamine | 0.8 | 24 | 8.2 | 8.8 | 7.6 | 7.0 | 6.4 | 4.6 | 2.5 |
| Ex. 3 | additive | Triethylamine | 0.8 | 14 | 6.4 | 6.4 | 6.0 | 5.8 | 4.0 | 3.7 | 1.7 |
| Ex. 4 | | Triethanolamine | 0.8 | 9 | 7.5 | 5.7 | 5.1 | 5.0 | 2.8 | 0.3 | 0.1 |
| Ex. 5 | | Polyvinylpyrrolidone | 1.0 | 13 | 8.7 | 5.2 | 3.6 | 2.8 | 0.5 | 0.3 | 0.1 |
| Ex. 6 | | Imidazole | 0.4 | 41 | 6.8 | 6.7 | 4.3 | 2.8 | 5.6 | 3.3 | 0.8 |
| Ex. 7 | | Imidazole | 1.6 | 41 | 6.0 | 6.2 | 6.2 | 6.6 | 7.5 | 4.8 | 0.8 |
| Ex. 8 | | Imidazole | 2.4 | 41 | 7.1 | 8.1 | 10.2 | 9.3 | 9.0 | 7.8 | 0.6 |
| Ex. 9 | | Polyvinylpyrrolidone | 0.5 | 13 | 7.8 | 7.1 | 3.2 | 2.5 | 1.9 | 1.1 | 0.5 |
| Ex. 10 | | Polyvinylpyrrolidone | 2.0 | 13 | 8.2 | 6.8 | 4.9 | 3.1 | 2.5 | 2.1 | 1.8 |
| Ex. 11 | | Imidazole · | 0.8 · | 33 | 7.3 | 6.6 | 5.9 | 5.7 | 4.3 | 4.2 | 2.8 |
| | | Trimethylamine | 0.8 | | | | | | | | |
| Comp. | Not added | | 0.0 | 0 | 7.8 | 5.5 | 4.2 | 1.1 | 0.5 | 0.1 | 0.1 |
| Ex. 1 | | | | | | | | | | | |
| Comp. | Polyacrylic acid-based thickener | | 1.0 | 0 | 7.6 | 3.5 | 2.8 | 1.5 | 0.4 | 0.1 | 0.1 |
| Ex. 2 | | | | | | | | | | | |
| Comp. | Nitrogen- | Polyvinylpyrrolidone | 5.0 | 13 | 7.1 | 5.2 | 4.2 | 1.5 | 0.2 | 0.1 | 0.0 |
| Ex. 3 | containing | | | | | | | | | | |
| | additive | | | | | | | | | | |

As presented in Tables 1 and 2 and FIGS. 1 and 2, in each of Examples, the contact impedance of the electrode sheet remained at 68 kΩ or less, and the adhesive strength was maintained to be 2.5 N/mm or greater, even when the water absorption rate was 30 wt % or greater. Conversely, in each of Comparative Examples, the contact impedance was greater than 30 kΩ, or the adhesive strength was 1.5 N/mm or less at the water absorption rate of 30 wt %.

Thus, it has been confirmed that, since the electrode sheet of each of Examples above includes a predetermined amount of the nitrogen-containing additive, the electrode sheet has low resistance, the contact impedance is improved, and excellent adhesion can be maintained even when the external moisture is absorbed during use. Therefore, even when the adhesive electrode for biosignal acquisition according to the present embodiment is adhered to the skin of a subject for a long time (e.g., 24 hours), the adhesive electrode biosignal acquisition can be effectively used for stably measuring biological information without troubling the subject continuously for a long time.

Note that embodiments of the present invention are, for example, as follows.
<1> An adhesive electrode for biosignal acquisition includes a conductive polymer, an aqueous emulsion adhesive, and a nitrogen-containing additive, wherein an amount of the nitrogen-containing additive is from 0.01 wt % to 3.0 wt % relative to a total amount of the adhesive electrode for biosignal acquisition.
<2> In the adhesive electrode for biosignal acquisition according to <1>, an amount of nitrogen in the nitrogen-containing compound is from 9 wt % to 45 wt % relative to a total amount of the nitrogen-containing compound.
<3> In the adhesive electrode for biosignal acquisition according to <1> or <2>, the nitrogen-containing additive is at least one component selected from the group consisting of an imidazole compound, low-molecular-weight amines, and polyvinylpyrrolidone.
<4> In the adhesive electrode for biosignal acquisition according to any one of <1> to <3>, the aqueous emulsion adhesive is an acrylic emulsion adhesive.
<5> In the adhesive electrode for biosignal acquisition according to <4>, the acrylic emulsion adhesive is a silane-based emulsion adhesive including a water-dispersible copolymer and an organic liquid component that is compatible with the water-dispersible copolymer.
<6> A biosensor includes the adhesive electrode for biosignal acquisition of any one of <1> to <5>.

The present application is based on and claims priority to Japanese Patent Application No. 2022-153422, filed in Japan Patent Office on Sep. 27, 2022, the contents of which are incorporated herein by reference.

1. An adhesive electrode for biosignal acquisition, comprising:
a conductive polymer;
an aqueous emulsion adhesive; and
a nitrogen-containing additive,
wherein an amount of the nitrogen-containing additive is from 0.01 wt % to 3.0 wt % relative to a total amount of the adhesive electrode for biosignal acquisition.

2. The adhesive electrode for biosignal acquisition according to claim 1,
wherein an amount of nitrogen in the nitrogen-containing additive is from 9 wt % to 45 wt % relative to a total amount of the nitrogen-containing additive.

3. The adhesive electrode for biosignal acquisition according to claim 1,
wherein the nitrogen-containing additive is at least one component selected from the group consisting of an imidazole compound, low-molecular-weight amines, and polyvinylpyrrolidone.

4. The adhesive electrode for biosignal acquisition according to claim 1,
wherein the aqueous emulsion adhesive is an acrylic emulsion adhesive.

5. The adhesive electrode for biosignal acquisition according to claim 4,
wherein the acrylic emulsion adhesive is a silane-based emulsion adhesive including a water-dispersible copolymer and an organic liquid component that is compatible with the water-dispersible copolymer.

6. A biosensor comprising:
the adhesive electrode for biosignal acquisition of claim 1.
